(19) United States
(12) Patent Application Publication
(10) Pub. No.: US 2008/0096969 A1
(43) Pub. Date: Apr. 24, 2008

(54) N alpha-(Menthanecarbonyl)amino acid amides and use thereof as physiological cooling active ingredients

(75) Inventor: Jakob LEY, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/873,715

(22) Filed: Oct. 17, 2007

Related U.S. Application Data
(60) Provisional application No. 60/829,953, filed on Oct. 18, 2006.

(51) Int. Cl. A61K 31/16; A61K 8/42; A61P 43/00; A61Q 19/00; C07C 233/57

(52) U.S. Cl. 514/616; 564/152

(57) ABSTRACT

The present invention relates to specific N^{α}-(menthanecarbonyl)amino acid amides of the formula (I):

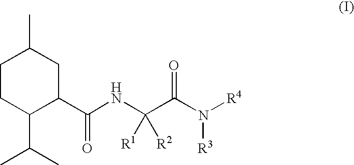

and mixtures thereof and to the use of the specific N^{α}-(menthanecarbonyl)amino acid amides and mixtures thereof as physiological cooling active ingredients.

### FIELD OF THE INVENTION

The invention relates to specific N^{α}-(menthanecarbonyl)amino acid amides and mixtures thereof which are capable of bringing about a physiological cooling action on the skin and/or a mucous membrane. It also relates to blends and preparations which contain the N^{α}-(menthanecarbonyl)amino acid amides in sufficient quantity for a cooling action to be produced on the skin and/or mucous membranes. It moreover relates to the use of the stated compounds as a cooling substance or for the production of a medicament and to a method for achieving a physiological cooling action on the skin and/or mucous membranes.

### BACKGROUND OF THE INVENTION

Physiological cooling active ingredients are often used to bring about a sensation of coolness on the skin or mucous membranes, for example on the mucous membranes in the oral, nasal and/or pharyngeal cavities, but without any physical cooling, such as occurs for example on solvent evaporation, actually occurring. Both individual components and mixtures may be used as physiological cooling active ingredients.

The best known cooling active ingredient is L-menthol, but this exhibits various disadvantages, for example a strong odor impression, elevated volatility and, at relatively high concentrations, a bitter and/or spicy hot intrinsic flavor. In certain aroma compositions, in particular those which do not tend towards a (pepper)mint aroma, the use of L-menthol may thus be undesirable.

Investigations have already been carried out which were directed towards strong cooling active ingredients without an aroma effect. DE 2 608 226 has accordingly described, for example, lactic acid esters of menthol(s) and DE 4 226 043 has described mixed carbonates with menthol(s) and polyols, which, while having a strong cooling action, on hydrolysis in aqueous media, may however give rise to the strong smelling menthol. Menthone ketals according to EP 0 507 190 B1 are strong cooling active ingredients, which, in acidic media, may however liberate menthone and due to the latter's aromatic action (strong intrinsic flavor and low threshold value) cannot be widely used.

While menthyl monoesters of diacids according to U.S. Pat. No. 5,725,865 and U.S. Pat. No. 5,843,466 are indeed interesting naturally occurring alternatives, in organoleptic testing they cannot achieve the strength of previously described cooling active ingredients. Moreover, being esters, they are also susceptible to hydrolysis.

While menthol polyol ethers, for example from EP 1 398 306, are indeed more resistant to hydrolysis, they provide a somewhat weaker cooling impression.

H. R. Watson, R. Hems, D. G. Rowsell and D. J. Spring, J. Soc. Cosmet. Chem. 1978, 29, 185-200 present the results of a study of approx. 1200 compounds, in which the compounds L-menthane carboxylic acid N-ethylamide (“WS3”) and in particular N^{α}-(L-menthanecarbonyl)glycine ethyl ester (“WS5”) were found to be the most strongly cooling active ingredients. The latter, while having a strong action, has the disadvantage of being susceptible to hydrolysis and, as a result, forming the corresponding free acid N-(L-menthanecarbonyl)glycine, which itself exhibits only a very weak cooling action. Despite the exhaustive investigations which have been described, a systematic prediction of the properties of potential cooling active ingredients, in particular regarding the bitterness thereof and/or the other trigeminal effects thereof, is not possible and has also not been described. Accordingly, while many molecules falling within the class of menthane carboxamides are indeed strongly cooling, they frequently simultaneously exhibit marked bitter notes (for example the menthane carboxylic acid N-(alkyloxyalkyl)amides according to JP 2004059474) or are additionally strongly irritant (WS5: N-[[5-methyl-2-(1-methylethyl)cyclohexyl]carbonyl]glycine ethyl ester, US 2005/0222256).

N^{α}-(Menthanecarbonyl) alkyloxyalkylamides have been described in JP 2004059474. These have at strong cooling action and elevated resistance to hydrolysis, but suffer the disadvantage of being strongly bitter and thus being unusable in foodstuffs and also in cosmetic products for facial care.

### SUMMARY OF THE INVENTION

The primary object of the present invention was therefore to provide novel compounds or mixtures of compounds which have a strong physiological cooling action, stability (resistance to hydrolysis) which is good and improved in comparison with known cooling active ingredients and which may be used as cooling substances (cooling active ingredients) in foodstuffs and/or products consumed for pleasure and/or oral care products and/or (oral) pharmaceutical preparations. The compounds or mixtures of compounds to be provided should preferably exhibit the weakest possible intrinsic flavor, in particular should taste only slightly or not at all bitter and exhibit the slightest possible irritancy.

### DETAILED DESCRIPTION OF THE PREFERRED EMBODIMENT

This primary object is achieved according to the invention by a compound (N^{α}-(menthanecarbonyl)amino acid amide) or a mixture of compounds of the general formula (I):

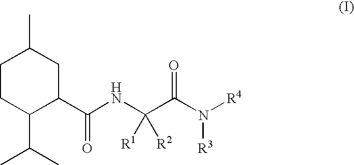

wherein
R¹ and R² in each case mutually independently mean hydrogen or a
   (a) linear, branched or cyclic,
   (b) saturated or unsaturated,
   (c) unsubstituted, mono- or polysubstituted hydrocarbon residue with
   (d) 1 to 5 carbon atoms, wherein
   (e) the hydrocarbon residues R¹ and R² may also be linked via a single bond or via an —O—, —S— or —NH— group and so preferably form a 3- to 7-membered ring,
      and
R³ and R⁴ in each case mutually independently mean hydrogen or a
   (a) linear, branched or cyclic,
   (b) saturated or unsaturated, hydrocarbon residue with
   (c) 1 to 10 carbon atoms, wherein
   (d) the hydrocarbon residues R³ and R⁴ may also be linked via a single bond or via an —O—, —S— or —NH— group and so preferably form a 3 to 7-membered heterocyclic ring,
      or
R³ and R⁴ mutually independently in each case mean hydrogen or a
   (a) linear or branched,
   (b) hydrocarbon residue substituted by an R⁵⁻X— group with
   (c) 1 to 10 carbon atoms, wherein each X, if present, in each case independently of the other X, if present,
   (d) represents oxygen, sulfur or an —NR— group,
      and
      wherein each R⁵ and each R⁶, if present, in each case mutually independently mean hydrogen or a
(a) linear, branched or cyclic,
(b) saturated or unsaturated, hydrocarbon residue with
(c) 1 to 5 carbon atoms,
   and
   wherein preferably the particular compound of the formula (I) individually or one of the compounds of the formula (I) in the mixture of compounds of the formula (I) independently of the other compounds of the formula (I) is present as an individual stereoisomer (enantiomer and diastereomer) or as a specific mixture of different stereoisomers.

In connection with the present application, specific mixtures are in particular mixtures in which an individual stereoisomer corresponding to the formula (I) constitutes 90 to 100 mol %, relative to the total number of the isomeric compounds belonging to this stereoisomer.

In the present document, a linear, branched or cyclic, saturated or unsaturated hydrocarbon residue with 1 to 5 carbon atoms, which may in turn be mono- or polysubstituted, in particular methyl, ethyl, propyl, 2-propyl, 2-methylprop-1-yl, 2-methylpropyl, 2-butyl, 2-methylbutyl residue, or a methyl residue, which may in turn be substituted by aryl residues, heterocyclyl residues, H₂N—, guanidino, (iso)urea, HO—, alkoxy, HS—, alkylthio, (substituted) sulfide, (substituted) disulfide, (substituted) phosphate, —COOH, —CONH₂, wherein the substituents, preferably together with the methyl residue, yield 4-aminobutyl, 3-guanidinopropyl, 3-aminopropyl, 3-ureidopropyl, indol-3-ylmethyl, 2-carboxyethyl, carboxymethyl, 2-(aminocarbonyl)ethyl, (aminocarbonyl)methyl, thiomethyl, 2-thioethyl, 2-methylthioethyl, hydroxymethyl, 1-hydroxyethyl, phenylmethyl and 4-hydroxyphenylmethyl.

In the present document, a linear, branched or cyclic, saturated or unsaturated hydrocarbon residue with 1 to 5 carbon atoms is preferably in particular methyl, ethyl, 2-propyl, cyclopropyl, propyl, butyl, cyclobutyl, 2-butyl, 2-methylprop-1-yl, 2-methylprop-2-yl, pentyl, cyclopentyl, 2-pentyl and 3-pentyl.

In the present document, a linear, branched or cyclic hydrocarbon residue with 1 to 10 carbon atoms is in particular methyl, ethyl, 2-propyl, cyclopropyl, propyl, butyl, cyclobutyl, 2-butyl, 2-methylprop-1-yl, 2-methylprop-2-yl, pentyl, cyclopentyl, 2-pentyl, 3-pentyl, hexyl, cyclohexyl, 2-hexyl and 3-hexyl, heptyl, octyl, isooctyl, nonyl, decyl.

In the present document, a linear or branched hydrocarbon residue with 1 to 10 carbon atoms substituted by an R⁵⁻X— group is in particular a hydrocarbon residue with 1 to 5 carbon atoms, for example methylene, ethylene, 1,2-propylene, 1,3-propylene, 1,2-butylene, 1,3-butylene, 1,4-butylene, 1,2-pentylene, 1,3-pentylene, 1,4-pentylene and 1,5-pentylene.

Preference is given to a compound according to the invention of the formula (I) or a mixture according to the invention of compounds of the formula (I):

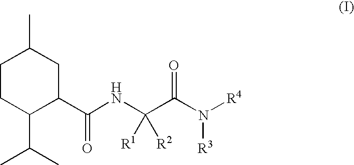

wherein R¹ means hydrogen
and/or
R means hydrogen or a
   (a) linear, branched or cyclic,
   (b) saturated or unsaturated,
   (c) unsubstituted, mono- or polysubstituted hydrocarbon residue with
   (d) 1 to 5 carbon atoms
      and/or
R³ and R⁴ in each case mutually independently mean hydrogen or a
   (a) linear, branched or cyclic, hydrocarbon residue with
   (b) 1 to 10 carbon atoms, wherein
   (c) the hydrocarbon residues R³ and R⁴ are preferably linked via a single bond or via an —O—, or —NH— group and so form an azirine, azetidine, pyrrolidine, imidazolidine, piperidine, pyrazolidine, diazane or morpholine ring
      or
R³ and R⁴ mutually independently in each case mean hydrogen or a
   (a) linear or branched,
   (b) hydrocarbon residue substituted by an R⁵⁻X— group with
   (c) 1 to 10 carbon atoms, wherein preferably each X, if present, in each case independently of the other X, if present,
   (d) represents oxygen, sulfur or an —NR⁶— group and wherein preferably each R⁶, if present, means hydrogen.

Further preference is given to a compound according to the invention or a mixture according to the invention

wherein the compound of the formula (I) (N-(menthanecarbonyl)amino acid amide) or a compound of the formula (I) in the mixture of compounds of the formula (I), independently of the other compounds of the formula (I), is 90-100 mol % defined, with regard to the stereochemistry thereof, in accordance with the formula (Ia)

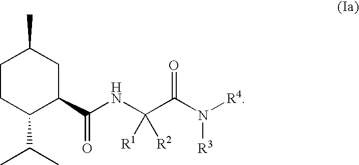

wherein
in the formula (Ia) the residues R¹, R², R³ and R⁴ in each case have the meaning of the corresponding residues of the formula (I)
and wherein preferably all the compounds of the formula (I) in the mixture are 90-100 mol % defined, with regard to the stereochemistry thereof, in accordance with the formula (Ia).

Particular preference is given to a compound according to the invention (N^{α}-(menthanecarbonyl)amino acid amide) of the formula (I) or a mixture according to the invention of compounds of the formula (I), wherein in the formula (I)

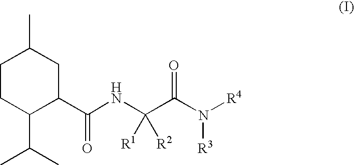

R¹ means hydrogen
   and/or
R² means hydrogen or a methyl, ethyl, propyl, 2-propyl, 2-methylpropyl, 2-butyl, 2-methylbutyl, 4-aminobutyl, 3-guanidinopropyl, 3-aminopropyl, 3-ureidopropyl, indol-3-ylmethyl, 2-carboxyethyl, carboxymethyl, 2-(aminocarbonyl)ethyl, (aminocarbonyl)methyl, thiomethyl, 2-thioethyl, 2-methylthioethyl, hydroxymethyl, 1-hydroxyethyl, phenylmethyl or 4-hydroxyphenylmethyl residue
   and/or
R³ means hydrogen or a methyl or ethyl residue,
   and/or
R⁴ means hydrogen or a methyl, ethyl, 2-propyl, cyclopropyl, propyl, butyl, cyclobutyl, 2-butyl, 2-methylprop-1-yl, 2-methylprop-2-yl, pentyl, cyclopentyl, 2- pentyl, 3-pentyl, hexyl, cyclohexyl, 2-hexyl and 3-hexyl, heptyl, octyl, isooctyl, nonyl or decyl residue,
   or
R⁴ means a methylene, ethylene, 1,2-propylene, 1,3-propylene, 1,2-butylene, 1,3-butylene, 1,4-butylene, 1,2-pentylene, 1,3-pentylene, 1,4-pentylene or 1,5-pentylene residue substituted by an R⁵⁻X— group, wherein X represents oxygen and R⁵ means hydrogen or a methyl or ethyl residue,
   and/or
   wherein preferably the compound or the compounds of the formula (I) are 90-100 mol % defined, with regard to the stereochemistry thereof, in accordance with the formula (Ia):

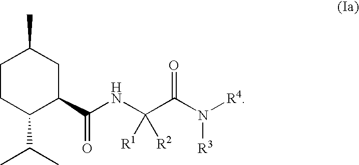

and wherein the particular residues R have the above-stated meaning and wherein preferably the compound or the compounds of the formula (I) are 90-100 mol % defined, with regard to the stereochemistry thereof, in accordance with the formula (Ia).

Very particular preference is given to a compound according to the invention or a mixture according to the invention of compounds of the formula (I) (N^{α}-(menthanecarbonyl)amino acid amides)

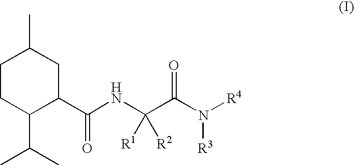

wherein
R¹ means hydrogen
   and/or
R² means hydrogen or a methyl, ethyl, propyl or 2-propyl residue
   and/or
R³ means hydrogen or a methyl or ethyl residue,
   and/or
R⁴ means hydrogen or a methyl, ethyl, 2-propyl, cyclopropyl, propyl, butyl, cyclobutyl, 2-butyl, 2-methylprop-1-yl, 2-methylprop-2-yl, pentyl, cyclopentyl, 2-pentyl, 3-pentyl, hexyl, cyclohexyl, 2-hexyl and 3-hexyl, heptyl, octyl, isooctyl, nonyl or decyl residue,
   or
R⁴ means a methylene, ethylene, 1,2-propylene or 1,3-propylene substituted by an R⁵⁻X— group,
   wherein X represents oxygen and
R⁵ means hydrogen or a methyl residue,
   and wherein preferably the N^{α}-(menthanecarbonyl)amino acid amides of the formula (I) according to the invention are 90 to 100 mol % defined, with regard to the stereochemistry thereof, in accordance with the formula (Ia)

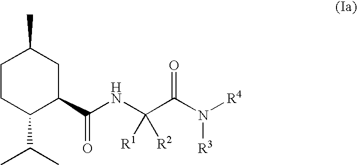

wherein the particular residues R in the formula (Ia) have the above-stated meaning.

Particularly preferred individual compounds which may be mentioned are:
L-N^{α}-(menthanecarboxyl)glycine-N-isobutylamide (compound 1)
L-N^{α}-(menthanecarboxyl)glycine-N,N-dimethylamide (compound 2)
L-N^{α}-(menthanecarboxyl)glycine-N-ethylamide (compound 3)
L-N^{α}-(menthanecarboxyl)glycine-N-ethanolamide (compound 4)
L-N^{α}-(menthanecarboxyl)-L-alanine-N-ethylamide (compound 5)

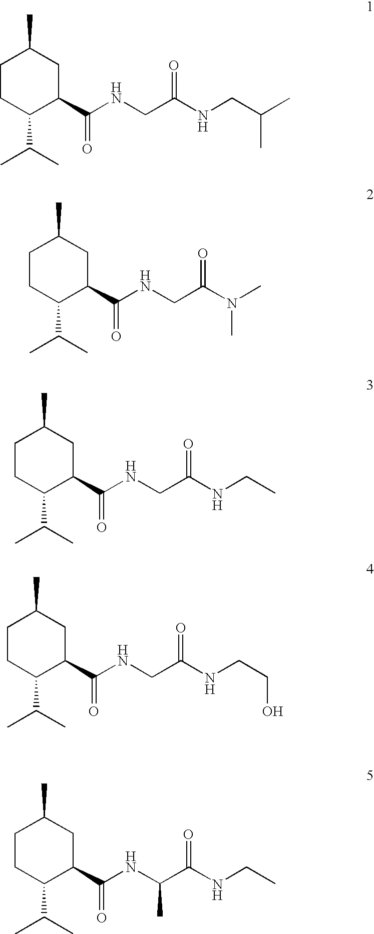

The invention is based on the surprising recognition that the compounds according to the invention, (N^{α}-(menthanecarbonyl)amino acid amide) of the formulae (I) and (Ia)) and mixtures thereof cause a strong and long-lasting sensation of coldness on the skin or mucous membrane, in particular on the mucous membranes of the oral, nasal and pharyngeal cavities. Said compounds here exhibit no other trigeminal effects such as spiciness, tingling or numbing and are not bitter. At the same time, within the bounds of conventional formulations and conditions of preparation, the compounds according to the invention are resistant to hydrolysis in the range from pH 1 to pH 12, in particular in the range from pH 4 to pH 9, in relation to preparations containing water, such that the compounds and mixtures according to the invention have a long storage life in preparations and the particular preparation itself in turn has a long storage life.

In connection with the present document, resistant to hydrolysis means that the investigated compounds are less than 10 mol % (are preferably not significantly) hydrolysed at 40° C., at pH 9 and/or pH 3, in daylight and at a water content of at least 5 wt. % after 6 months and furthermore preferably exhibit less than 10% (once again preferably no significant) hydrolysis reactions under the production conditions conventional for the preparations stated in the present document.

The invention relates to also a blend consisting of/comprising
(a) a compound according to the invention or a mixture according to the invention of compounds
   and
(b) one or more further substances with a physiological cooling action, wherein the further substance or one, several or all of the further substances (i) cause(s) a flavor effect or (ii) do(es) not cause a flavor effect,
   and/or
(c) one or more aroma substances without a physiological cooling action
   and/or
(d) one or more substances without a physiological cooling action which have a trigeminal or salivatory action.

A particularly preferred blend according to the invention is one comprising as constituent (b) one or more further substances with a physiological cooling action, these causing no flavor effect and no aroma action, but instead merely a cooling action (substantially) without any further organoleptic effect. This prevents the aroma profile of the blend being for example shifted towards “mint” (peppermint).

A very particularly preferred blend according to the invention is one comprising as constituent (c) one or more aroma substances without a physiological cooling action and/or as constituent (d) one or more compounds which mutually independently or jointly additionally cause a flavor-modulating effect and/or a trigeminal and/or a salivatory stimulus, wherein the trigeminal stimulus preferably does not constitute a physiological cooling action. In particular, such blends according to the invention which simultaneously contain the latter-stated constituents (c) and (d) have a pleasant cooling action and a balanced organoleptic profile with a simultaneously elevated impact, i.e. an elevated initial flavor impression.

The one or more further substances with a physiological cooling action which may be used as constituent (b) in a blend according to the invention are here preferably selected from the following list: menthol and menthol derivatives (for example L-menthol, D-menthol, racemic menthol, isomenthol, neoisomenthol, neomenthol), menthyl ethers (for example (I-menthoxy)-1,2-propanediol, (I-menthoxy)-2-methyl-1,2-propanediol, 1-menthyl methyl ether), menthyl esters (for example menthyl formate, menthyl acetate, menthyl isobutyrate, menthyl lactate, L-menthyl L-lactate, L-menthyl D-lactate, menthyl (2-methoxy)acetate, menthyl (2-methoxyethoxy)acetate, menthyl pyroglutamate), menthyl carbonates (for example menthyl propylene glycol carbonate, menthyl ethylene glycol carbonate, menthyl glycerol carbonate or mixtures thereof), the semi-esters of menthols with a dicarboxylic acid or the derivatives thereof (for example monomenthyl succinate, monomenthyl glutarate, monomenthyl malonate, O-menthyl succinic acid ester N,N-(dimethyl)amide, O-menthyl succinic acid ester amide), menthane carboxamides other than those stated in the present invention (for example menthane carboxylic acid N-ethylamide [WS3], N^{α}-(menthanecarbonyl)glycine ethyl ester [WS5], menthane carboxylic acid N-(4-cyanophenyl)amide, menthane carboxylic acid N-(alkoxyalkyl)amides), menthone and menthone derivatives (for example L-menthone glycerol ketal), 2,3-dimethyl-2-(2-propyl)-butanoic acid derivatives (for example 2,3-dimethyl-2-(2-propyl)-butanoic acid N-methylamide [WS23]), isopulegol or the esters thereof (I—(−)-isopulegol, I—(−)-isopulegol acetate), menthane derivatives (for example p-menthane-3,8-diol), cubebol or synthetic or natural blends containing cubebol, pyrrolidone derivatives of cycloalkyldione derivatives (for example 3-methyl-2(1-pyrrolidinyl)-2-cyclopenten-1-one) or tetrahydropyrimidin-2-one (for example icilin or related compounds, as described in WO 2004/026840).

The one or more further substances with a physiological cooling action which may be used as constituent (b) of a blend according to the invention are preferably substances which at least substantially cause a physiological cooling action without simultaneously causing a flavor action. Such preferred substances are: menthyl ethers (for example (I-menthoxy)-1,2-propanediol, (I-menthoxy)-2-methyl-1,2-propanediol), relatively highly polar menthyl esters (for example menthyl lactate, L-menthyl L-lactate, L-menthyl D-lactate, menthyl (2-methoxy)acetate, menthyl (2-methoxyethoxy)acetate, menthyl pyroglutamate), menthyl carbonates (for example menthyl propylene glycol carbonate, menthyl ethylene glycol carbonate, menthyl glycerol carbonate), the semi-esters of menthol with a dicarboxylic acid or the derivatives thereof (for example monomenthyl succinate, monomenthyl glutarate, monomenthyl malonate, O-menthyl succinic acid ester N,N-(dimethyl)amide, O-menthyl succinic acid ester amide), menthane carboxamides not according to the invention (for example menthane carboxylic acid N-ethylamide [WS3], N^{α}-(menthanecarbonyl)glycine ethyl ester [WS5], menthane carboxylic acid N-(4-cyanophenyl)amide, menthane carboxylic acid N-(alkoxyalkyl)amides), menthone derivatives (for example L-menthone glycerol ketal), 2,3-dimethyl-2-(2-propyl)-butanoic acid derivatives, (for example 2,3-dimethyl-2-(2-propyl)-butanoic acid N-methylamide), pyrrolidone derivatives of cycloalkyldione derivatives (for example 3-methyl-2(1-pyrrolidinyl)-2-cyclopenten-1-one) or tetrahydropyrimidin-2-one (for example icilin or related compounds which are described in WO 2004/026840).

Specific blends of menthane carboxamides with cooling active ingredients such as acyclic carboxamides and L-menthyl lactate and optionally further cooling active ingredients or trigeminal stimulants are described for example in US 2005/0117811; said document, however, makes no mention of using the menthane carboxylic acid N^{α}-(menthanecarbonyl)amino acid amides according to the invention (compounds of the formulae (I) and (Ia)). The substituents stated in US 2005/0117811 also provide no indication regarding the use according to the invention.

Preferred aroma substances without a physiological cooling action are those aroma substances which, in addition to their actual odorous aroma value, also cause a flavor impression, a flavor-modulating effect or a trigeminal, but non-cooling or also a salivatory stimulus. Preferred flavor impressions are sweet, umami, bitter, salty and sour; preferred flavor-modulating effects are bitter-masking, umami-enhancing, sweet-enhancing, salt-enhancing and sour-masking effects; preferred trigeminal stimuli are spiciness, heat, tingling and pungency. Particularly preferred aroma substances without a physiological cooling action are for example pellitorines according to WO 04 000,787 or US 2004/0241312 and alkamides according to DE 103 51 422.

The compounds according to the invention of the formula (I) or the corresponding mixtures are preferably synthesized by reaction with the corresponding N-(menthanecarbonyl)amino acid, a corresponding N^{α}-(menthanecarbonyl)amino acid chloride or another corresponding activated N^{α}-(menthanecarbonyl)amino acid derivative with a corresponding amine or a corresponding salt.

An N^{α}-(menthanecarbonyl)amino acid comprising more than 90 wt. %, preferably more than 95 wt. %, of (L)-N^{α}-(menthanecarbonyl)amino acid or an (L)-N^{α}-(menthanecarbonyl)amino acid chloride produced therefrom or another activated (L)-N⁶⁰-(menthanecarbonyl)amino acid derivative produced therefrom is preferably used. Synthesis may proceed in accordance with per se known acylation methods.

The invention furthermore also relates to a corresponding method for the production of a compound according to the invention or for the production of a mixture of compounds according to the invention.

A preferred synthetic pathway may be illustrated with reference to the following reaction scheme, which gives rise to a compound of the formula (I). A corresponding synthetic pathway is preferred for the production of a compound of the formula (Ia).

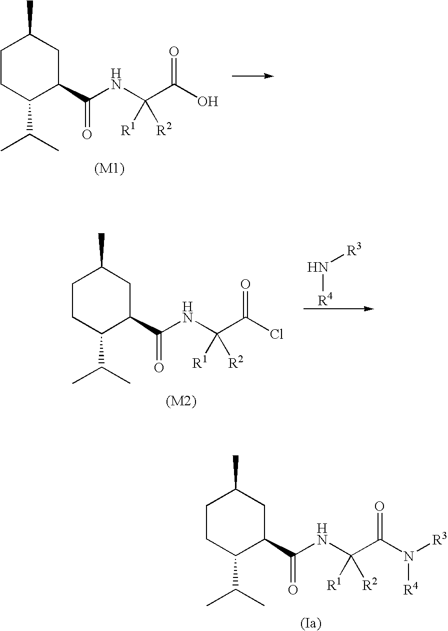

The L-N^{α}-(menthanecarbonyl)amino acid (M1) may here be converted, for example using known methods, into the corresponding acid chloride (M2) by means of SOCl₂, (COCl)₂ or PCl₃.

This reaction may optionally proceed in the presence of other (auxiliary) substances or additives, for example in the presence of
(i) one or more solvents or diluents (for example water, water/1,4-dioxane mixtures, tetrahydrofuran, water/tetrahydrofuran mixtures, other ethers, chloroform, methylene chloride, ethyl acetate, acetone, acetone/water mixtures, alkanes, alcohols) and/or
(ii) inorganic or organic auxiliary bases (for example triethylamine, other trialkylated amines, carbonates, hydroxides, basic oxides or hydrogencarbonates of alkali and/or alkaline earth metals, basic ion exchangers), and/or
(iii) phase-transfer catalysts (for example peralkylated/perarylated ammonium salts or phosphonium salts, crown ethers), and/or
(iv) enzymes and/or suitable microorganisms (in particular hydrolytic enzymes such as lipases, amidases, peptidases).

The crude synthesis products are preferably purified or concentrated by physical, optionally also enantioselective or enantiospecific separation methods, for example extraction, partition methods, crystallization, distillation, chromatography, sublimation, steam distillation, reverse osmosis, permeation or the like, the separation method preferably being selected such that, after the separation operation, the stereochemistry of the N^{α}-(menthanecarbonyl)amino acid of the formula (Ia) according to the invention (to the extent that the formula (Ia) defines the stereochemistry) corresponds to a proportion of 90-100 mol %, preferably 95-100 mol %, relative to the total quantity of the compounds of the formula (I) present in the purified product.

The invention furthermore also relates to preparations consumed for nutrition or for pleasure or used for oral hygiene or to pharmaceutical or cosmetic preparations, which preparations, in order to achieve a physiological cooling action on the skin and/or mucous membranes, comprise a sufficient quantity (a) of a compound according to the invention or a mixture according to the invention (preferably in a development which is stated to be preferred) or (b) of a blend according to the invention (preferably in a development which is stated to be preferred). In particular, the quantity of the compound, mixture or blend used should be sufficient to achieve a physiological cooling action on the mucous membranes in the oral, nasal and/or pharyngeal cavities.

Preferred preparations according to the invention comprise conventional basic materials, auxiliary substances and additives for preparations consumed for nutrition or for pleasure or used for oral hygiene or for pharmaceutical or cosmetic preparations. Preferred preparations according to the invention contain 0.0001 wt. % to 20 wt. %, preferably 0.0001 to 10 wt. %, particularly preferably 0.001 wt. % to 0.5 wt. % of compounds of the formula (I), relative to the total weight of the preparation. Further constituents, in particular compounds of the formula (Ia) and constituents (b), (c) and/or (d) and further conventional basic materials, auxiliary substances and additives may be present in quantities of 0.0000001 to 99.99 wt. %, preferably of 10 to 80 wt. %, relative to the total weight of the preparation. The preparations according to the invention may furthermore contain water in a quantity of up to 99.99 wt. %, preferably of 5 to 80 wt. %, relative to the total weight of the preparation.

The preparations consumed for nutrition or for pleasure are for example bakery products (for example bread, dry biscuits, cakes, other pastry products), confectionery (for example chocolates, chocolate bar products, other bar products, fruit gums, hard and soft caramels, chewing gum), alcoholic or non-alcoholic beverages (for example coffee, tea, wine, beverages containing wine, beer, beverages containing beer, liqueurs, spirits, brandies, fruit-containing carbonated beverages, isotonic beverages, soft drinks, nectars, fruit and vegetable juices, fruit or vegetable juice preparations), instant beverages (for example instant cocoa beverages, instant tea beverages, instant coffee beverages), meat products (for example ham, fresh or cured sausage preparations, spiced or marinated fresh or cured meat products), eggs or egg products (dried egg, egg white, egg yolk), cereal products (for example breakfast cereals, muesli bars, precooked ready rice products), dairy products (for example milk beverages, milk ice cream, yogurt, kefir, curd cheese, soft cheese, hard cheese, dried milk powder, whey, butter, buttermilk), fruit preparations (for example jams, fruit ice cream, fruit sauces, fruit fillings), vegetable preparations (for example ketchup, sauces, dried vegetables, deep-frozen vegetables, precooked vegetables, preserved vegetables), snack articles (for example baked or fried potato chips or potato dough products, maize- or peanut-based extrudates), fat- or oil-based products or emulsions thereof (for example mayonnaise, remoulade, dressings), other ready-to-serve meals and soups (for example dried soups, instant soups, precooked soups), spices, seasoning mixtures and in particular powdered seasonings, which are for example used in snack food applications. The preparations for the purposes of the invention may also be used as semifinished products for the production of further preparations consumed for nutrition or for pleasure. The preparations for the purposes of the invention may also be nutritional supplements in the form of capsules, tablets (uncoated and coated tablets, for example coatings resistant to gastric juices), sugar-coated tablets, granules, pellets, mixtures of solids, dispersions in liquid phases, as emulsions, as powders, as solutions, as pastes or as other swallowable or chewable preparations.

Preparations for oral hygiene purposes are in particular dental care products such as toothpastes, tooth gels, tooth powders, mouthwashes, chewing gum and other oral care products.

Dental care products (as the basis for preparations for oral care purposes) which contain the compounds, mixtures or blends according to the invention generally comprise an abrasive system (abrasive or polishing agent), such as for example silicas, calcium carbonates, calcium phosphates, aluminum oxides and/or hydroxyapatites, surface-active substances such as for example sodium lauryl sulfate, sodium lauryl sarcosinate and/or cocamidopropyl betaine, humectants such as for example glycerol and/or sorbitol, thickeners, such as for example carboxymethylcellulose, polyethylene glycols, carrageenan and/or Laponite®, sweeteners, such as for example saccharin, sodium cyclamate, sucralose, acesulfame K or sugar alcohols, flavor-correcting agents for unpleasant flavor impressions such as for example hydroxyflavanones according to US 2002/0188019, flavor-correcting agents for further, generally not unpleasant flavor impressions, flavor-modulating substances (for example inositol phosphate, nucleotides such as guanosine monophosphate, adenosine monophosphate or other substances such as sodium glutamate or 2-phenoxypropionic acid), cooling active ingredients such as for example menthol, menthol derivatives (for example L-menthol, L-menthyl lactate, L-menthyl alkylcarbonates, menthone ketals, menthane carboxamides), 2,2,2-trialkylacetamides (for example 2,2-diisopropyl propionic acid methylamide), icilin and icilin derivatives, stabilizers and active ingredients, such as for example sodium fluoride, sodium monofluorophosphate, tin difluoride, quaternary ammonium fluorides, zinc citrate, zinc sulfate, tin pyrophosphate, tin dichloride, blends of different pyrophosphates, triclosan, cetylpyridinium chloride, aluminum lactate, potassium citrate, potassium nitrate, potassium chloride, strontium chloride, hydrogen peroxide, aromas and/or sodium bicarbonate or flavor-correcting agents.

Chewing gums (as a further example of the preparations for oral care purposes) which contain the compounds, mixtures or blends according to the invention generally comprise a chewing gum base, i.e. a chewable mass which becomes plastic on chewing, sugars of various kinds, sugar substitutes, other sweet-tasting substances, sugar alcohols, flavor-correcting agents for unpleasant flavor impressions, other flavor modulators for further, generally not unpleasant flavor impressions, flavor-modulating substances (for example inositol phosphate, nucleotides such as guanosine monophosphate, adenosine monophosphate or other substances such as sodium glutamate or 2-phenoxypropionic acid), humectants, thickeners, emulsifiers, aromas and stabilizers or flavor-correcting agents.

Pharmaceutical preparations according to the invention which are preferred for the purposes of the invention are oral preparations, which for example assume the form of capsules, tablets (uncoated and coated tablets, for example coatings resistant to gastric juices), sugar-coated tablets, granules, pellets, mixtures of solids, dispersions in liquid phases, as emulsions, as powders, as solutions, as pastes or as other swallowable or chewable preparations and are used as prescription-only, drugstore-only or other medicaments or as nutritional supplements.

Cosmetic preparations according to the invention may for example be present in one of the following forms: soap, synthetic detergent, a liquid washing, shower or bath preparation, emulsion (as a solution, dispersion, suspension; cream, lotion or milk depending on the production method and constituents of the “water-in-oil” (W/O), “oil-in-water” (O/W) or multiple emulsion, PIT emulsion, emulsion foam, microemulsion, nanoemulsion or Pickering emulsion type), ointment, paste, gel (including hydrogel, hydrodispersion gel, oleogel), oil, toner, balsam, serum, powder, eau de toilette, toilet water, eau de cologne, perfume, wax, as a stick, roll-on, (pump) spray, aerosol (foaming, non-foaming or post-foaming), as a foot care product (including keratolytics, deodorant), beard shampoo or care preparations, insect-repellent product, sunscreen product, aftersun preparation, shaving preparation (for example shaving foams, soaps or gels) or aftershave preparation (balm, lotion), depilatory product, hair care product such as for example shampoo (including 2-in-1 shampoo, antidandruff shampoo, baby shampoo, shampoo for a dry scalp, shampoo concentrate), conditioner, hair tonic, hair water, hair rinse, hairdressing cream, pomade, permanent wave and setting lotion, hair smoothing product (detangling product, relaxer), hair strengthener, styling aid (for example gel or wax), blonding product, hair lightener, hair conditioner, hair foam, hair toning product, hair dyes (for example temporary, substantive, semipermanent, permanent hair dyes), nail care products such as for example nail polish and nail polish remover, deodorant and/or antiperspirant, mouthwash, water pick, makeup, makeup remover, eye care preparation, lip cosmetics, lip care preparation, decorative cosmetics (for example powder, eye shadows, kohl pencil, lipstick), bath articles (for example capsules) or mask.

Preparations according to the invention which comprise compounds according to the invention, a mixture according to the invention, or a blend according to the invention are preferably produced by incorporating the compound, the mixture or the blend, for example a blend comprising a solid or liquid carrier in addition to a compound according to the invention, into a base preparation. Advantageously, blends according to the invention, which are initially in solution form and comprise a compound according to the invention, are converted into a solid preparation by spray drying.

According to an alternative, preferred embodiment, preparations according to the invention may be produced by initially incorporating the compounds, mixtures or blends according to the invention, optionally with further constituents of the preparation according to the invention, into emulsions, into liposomes, for example starting from phosphatidyl choline, into microspheres, into nanospheres or also into capsules, granules or extrudates prepared from a matrix suitable for foodstuffs and products consumed for pleasure, for example prepared from starch, starch derivatives (for example modified starch), cellulose or cellulose derivatives (for example hydroxypropylcellulose), other polysaccharides (for example dextrin, alginate, curdlan, carageenan, chitin, chitosan, pullulan), natural fats, natural waxes (for example beeswax, carnauba wax), prepared from proteins, for example gelatin or other natural products (for example shellac) or non-natural matrix materials (such as polyurea). In said embodiment, depending on the matrix, the products may be treated by spray drying, spray granulation, melt granulation, coacervation, coagulation, extrusion, melt extrusion, emulsion methods, coating or other suitable encapsulation methods and optionally a suitable combination of the above-stated methods.

In a further preferred production method, the compounds, mixtures or blends according to the invention are initially complexed with one or more suitable complexing agents, for example with cyclodextrins or cyclodextrin derivatives, preferably alpha-, beta- or gamma-cyclodextrin, and in used in this complexed form.

A particularly preferred preparation according to the invention is one in which the matrix is selected such that the compounds, mixtures or blends according to the invention, in particular blends comprising further cooling active ingredients and/or aromas, are released from the matrix in delayed manner, such that a long-lasting cooling action is achieved.

Further constituents for preparations consumed for nutrition or for pleasure according to the invention which may be used are conventional basic materials, auxiliary substances and additives for foodstuffs or products consumed for pleasure, for example water, mixtures of fresh or processed, plant or animal basic or raw materials (for example raw, roasted, dried, fermented, smoked and/or boiled meat, bone, cartilage, fish, vegetables, fruit, herbs, nuts, vegetable or fruit juices or pastes or mixtures thereof), digestible or non-digestible carbohydrates (for example sucrose, maltose, fructose, glucose, dextrins, amylose, amylopectin, inulin, xylans, cellulose, tagatose), sugar alcohols (for example sorbitol, erythritol), natural or hardened fats (for example tallow, lard, palm fat, coconut oil, hardened vegetable fat), oils (for example sunflower oil, peanut oil, maize germ oil, olive oil, fish oil, soya oil, sesame oil), fatty acids or the salts thereof (for example potassium stearate), proteinogenic or non-proteinogenic amino acids and related compounds (for example γ-aminobutyric acid, taurine), peptides (for example glutathione), native or processed proteins (for example gelatin), enzymes (for example peptidases), nucleic acids, nucleotides, flavor-correcting agents for unpleasant flavor impressions, further flavor-modulators for further generally not unpleasant flavor impressions, other flavor-modulating substances (for example inositol phosphate, nucleotides such as guanosine monophosphate, adenosine monophosphate or other substances such as sodium glutamate or 2-phenoxypropionic acid), emulsifiers (for example lecithins, diacylglycerols, gum arabic), stabilizers (for example carrageenan, alginate), preservatives (for example benzoic acid, sorbic acid), antioxidants (for example tocopherol, ascorbic acid), chelating agents (for example citric acid), organic or inorganic acidulants (for example malic acid, acetic acid, citric acid, tartaric acid, phosphoric acid), bitter substances (for example quinine, caffeine, limonene, amarogentin, humolone, lupolone, catechins, tannins), mineral salts (for example sodium chloride, potassium chloride, magnesium chloride, sodium phosphates), substances preventing enzymatic browning (for example sulfite, ascorbic acid), essential oils, plant extracts, natural or synthetic dyes or coloring pigments (for example carotenoids, flavonoids, anthocyans, chlorophyll and the derivatives thereof), spices, trigeminally active substances or plant extracts containing such trigeminally active substances, synthetic, natural or nature-identical aroma substances or odoriferous substances and flavor-correcting agents.

Another aspect of the present invention relates to the use
(a) of a compound according to the invention or of a mixture according to the invention (as defined above, preferably in an above-described preferred development),
(b) of a blend according to the invention (as described above, preferably in a development described as being preferred) or
(c) of a preparation according to the invention (as described above, preferably in a development which is stated to be preferred),
   for producing a cooling action on the skin or a mucous membrane
(i) for purposes other than therapeutic or
(ii) for producing a medicament.

The compounds, mixtures and/or blends according to the invention are preferably used to produce a medicament which serves to combat or alleviate coughs, colds, symptoms of oral, nasal, throat or pharyngeal inflammation, sore throat or hoarseness.

A further aspect of the present invention relates to a method for achieving a physiological cooling action on the skin and/or a mucous membrane. Such a method according to the invention may be carried out for therapeutic or non-therapeutic (for example cosmetic) purposes and comprises the following step:
application of a quantity sufficient for achieving a physiological cooling action
(i) of a compound according to the invention or of a mixture according to the invention (as defined above, preferably in an above-described preferred development),
(ii) of a blend according to the invention (as described above, preferably in a development described as being preferred) or
(iii) of a preparation according to the invention (as described above, preferably in a development which is stated to be preferred),
   onto the skin and/or a mucous membrane.

A further aspect of the invention relates to the use of preparations according to the invention containing a compound according to the invention, a mixture according to the invention or a blend according to the invention, preferably a blend according to the invention which comprises one or more aroma substances and/or one or more further cooling active ingredients (cooling active ingredients which are not a compound of the general formula (I)), as semifinished products (“aroma blends”) for aromatizing finished products produced using the semifinished products.

Further aspects of the present invention emerge From the following Examples and the appended claims.

### EXAMPLES

The Examples merely serve to illustrate the invention without thereby limiting it. Unless otherwise stated, all stated values relate to weight.

### Example 1

### Synthesis of L-N^{α}-(menthanecarboxyl)glycine-N-isobutylamide (compound 1)

L-N^{α}-(Menthanecarboxyl)glycine ethyl ester (“WS 5”) was reacted with isobutylamine in toluene at 56° C. with the assistance of Chirazyme L2c2. After filtration, evaporation and chromatographic purification, it proved possible to obtain compound 1 as a crystalline, colorless pure substance.

¹H-NMR (400 MHz, CDCl₃, TMS): δ=6.82 (1H, br s, NH), 6.72 (1H, br s, NH), 3.92 (2H, d, 5.3 Hz), 3.08 (2H, dd, 6.7 Hz, 6.1 Hz), 2.13 (1H, ddd, 11.7 Hz, 11.4 Hz, 3.4 Hz), 1.82-1.62 (5H, m), 1.58-1.49 (1H, m), 1.42-1.3 (1H, m), 1.26-1.16 (1H, m), 1.08-0.8 (2H, m), 0.92 (6H, d, 6.7 Hz), 0.89 (3H, d, 6.5 Hz), 0.89 (3H, d, 6.9 Hz), 0.78 (3H, d, 6.9 Hz) ppm. ¹³C-NMR (100 MHz, CDCl₃, TMS): δ=176.88 (C), 169.31 (C), 49.27 (CH), 46.94 (CH₂), 44.33 (CH), 43.64 (CH₂), 39.45 (CH₂), 34.57 (CH₂), 32.25 (CH), 28.81 (CH), 28.48 (CH), 23.81 (CH), 22.31 (CH₂), 21.41 (CH₃), 20.10 (2×CH₃), 16.05 (CH₃) ppm. MS (EI): m/z=296 (M⁺, 50%), 224 (50%), 197 (45%), 167 (50%), 139 (50%), 131 (100%), 83 (80%)

### Example 2

### Synthesis of L-N^{α}-(menthanecarboxyl)glycine-N-ethylamide (compound 3)

L-N^{α}-(Menthanecarboxyl)glycine ethyl ester (“WS 5”) was saponified with KOH in water, the crude product was converted into the acid chloride with thionyl chloride and the product obtained by evaporation was reacted with ethylamine hydrochloride.

### Example 3

### Synthesis of L-N^{α}-(menthanecarboxyl)-L-alanine-N-ethylamide (compound 5)

In a manner similar to compound 3, compound 5 was obtained starting from L-N^{α}-(menthanecarboxyl)-L-alanine ethyl ester.

### Example of Application 1

### Cooling Action

The compounds were tested for their organoleptic properties, in particular their cooling action. To this end, they were dissolved, in each case in a specific final concentration, in a mass prepared from sucrose (saccharose) and water (confectioner's fondant, supplier Nordzucker A G, Nordstemmen) and evaluated by a panel of experts. Sensory impressions were rated and the cooling action was assessed on a scale from 1 (no cooling action) to 9 (extremely strong cooling action).

Profile of L-N^{α}-(menthanecarboxyl)glycine-isobutylamide (Example 1) at a concentration of 0.05 wt. %, relative to the overall preparation: very slightly bitter, cooling action 5

### Example of Application 2

### Aroma Blend for Achieving a Cooling Action

| Constituent | Proportion in wt. % |
|---|---|
| L-N^{α}-(Menthanecarboxyl)glycine-N-ethylamide | 25 |
| from Example 3 | |
| L-Menthyl lactate (Frescolat ML, Symrise) | 65 |
| O-L-Menthyl-O′-(2-hydroxyethyl) carbonate | 10 |
| (Frescolat MGC, Symrise) | |

A strongly cooling, but otherwise virtually flavorless and odorless aroma blend which is liquid at room temperature (20° C.) is obtained by blending the components.

### Example of Application 3

### Aroma Blend for Achieving a Cooling Action

| Constituent | Proportion in wt. % |
|---|---|
| L-N^{α}-(Menthanecarboxyl)glycine-N-isobutylamide | 7.5 |
| from Example 1 | |
| L-Menthane carboxylic acid N-ethylamide | 5 |
| (WS 3, for example Millennium) | |
| L-Menthyl lactate (Frescolat ML, Symrise) | 32.5 |
| O-L-Menthyl-O′-(2-hydroxyethyl) carbonate | 5 |
| (Frescolat MGC, Symrise) | |
| Propylene glycol | 50 |

A strongly cooling, but otherwise virtually flavorless and odorless aroma blend which is liquid at room temperature (20° C.) is obtained by blending the components.

### Example of Application 4

### Aroma Blend for Achieving an Aromatizing and Cooling Action

| Constituent | Proportion in wt. % |
|---|---|
| L-N^{α}-(Menthanecarboxyl)glycine-N-ethylamide | 15 |
| from Example 3 | |
| Peppermint oil | 10 |
| L-Menthyl lactate (Frescolat ML, Symrise) | 65 |
| O-L-Menthyl-O′-(2-hydroxyethyl) carbonate | 10 |
| (Frescolat MGC, Symrise) | |

A strongly cooling aroma blend with a strong odor of peppermint is obtained by blending the components.

### Example of Application 5

### Aroma Blend for Achieving a Cooling Action with a Simultaneous Tingling Effect

| Constituent | Proportion in wt. % |
|---|---|
| L-N^{α}-(Menthanecarboxyl)glycine-N-ethylamide | 15 |
| from Example 3 | |
| Solution of 10 wt. % pellitorine in propylene | 10 |
| glycol/peppermint oil 1:1 | |
| L-Menthyl lactate (Frescolat ML, Symrise) | 65 |
| O-L-Menthyl-O′-(2-hydroxyethyl) carbonate | 10 |
| (Frescolat MGC, Symrise) | |

A strongly cooling aroma blend which stimulates salivation and causes a tingling effect is obtained by blending the components.

### Example of Application 6

### Use in the Form of an Aroma Blend in a Toothpaste

| | | Quantity used in |
|---|---|---|
| Part | Constituent | wt. % |
| A | Demineralized water | 22.00 |
| | Sorbitol (70%) | 45.00 |
| | Solbrol ® M, sodium salt (Bayer AG, | 0.15 |
| | p-hydroxybenzoic acid alkyl ester) | |
| | Trisodium phosphate | 0.10 |
| | Saccharin, 450x | 0.20 |
| | Sodium monofluorophosphate | 1.12 |
| | Polyethylene glycol 1500 | 5.00 |
| B | Sident 9 (abrasive silicon dioxide) | 10.00 |
| | Sident 22 S (thickening silicon dioxide) | 8.00 |
| | Sodium carboxymethylcellulose | 0.90 |
| | Titanium dioxide | 0.50 |
| C | Demineralized water | 4.53 |
| | Sodium lauryl sulfate | 1.50 |
| D | Aroma blend from Example of application 2 | 1 |

The constituents of parts A and B were in each case individually premixed and in each case thoroughly stirred under a vacuum at 25-30° C. for 30 minutes. Part C was premixed and added to A and B; D was added and the blend was thoroughly stirred under a vacuum at 25-30° C. for a further 30 minutes. After relieving the vacuum, the toothpaste was ready and could be packaged.

A distinct cooling action could be identified when using the resultant toothpaste.

### Example of Application 7

### Use as Cooling Active Ingredient in a Sugar-Free Chewing Gum

| | | Quantity |
|---|---|---|
| | | used in |
| Part | Constituent | wt. % |
| A | Chewing gum base, company “Jagum T” | 30.00 |
| B | Powdered sorbitol | 39.00 |
| | Isomalt ® (Palatinit GmbH) | 9.50 |
| | Xylitol | 2.00 |
| | Mannitol | 3.00 |
| | Aspartame ® | 0.10 |
| | Acesulfame ® K | 0.10 |
| | Emulgum ® (Colloides Naturels, Inc.) | 0.30 |
| C | Sorbitol, 70% | 14.00 |
| | Glycerol | 1.00 |
| D | Spearmint/peppermint/eucalyptus aroma, | 1 |
| | containing 5 wt. % L-N^{α}-(menthanecarboxyl)glycine- | |
| | N-isobutylamide from Example 1 | |

Parts A to D were mixed and vigorously kneaded. The crude mixture was processed into ready-to-use chewing gum, for example in the form of thin strips.

A distinct cooling action could be identified when using the resultant chewing gum.

### Example of Application 8

### Use as Cooling Active Ingredient in a Mouthwash

| | | Quantity used in |
|---|---|---|
| Part | Constituent | wt. % |
| A | Ethanol | 10.00 |
| | Cremophor ® CO 40 (BASF, detergent) | 1.00 |
| | Benzoic acid | 0.12 |
| | Peppermint/lemon balm aroma containing | 0.25 |
| | 0.4 wt. % pellitorine and 10 wt. % L-N^{α}- | |
| | (menthanecarboxyl)-glycine-N-isobutylamide | |
| | from Example 1 | |
| B | Demineralized water | 83.46 |
| | Sorbitol, 70% | 5.00 |
| | Sodium saccharin 450 | 0.07 |
| | L-Blue 5000 e.c., 1% in water (dye) | 0.10 |

The constituents of parts A and B were in each case individually mixed. Part B was slowly stirred into part A until the blend was homogeneous.

A distinct cooling action could be identified when using the resultant mouthwash.

### Example of Application 9

### Throat Candies with Liquid/Viscous Core Filling (Centre-Filled Hard Candy)

| | I (wt. %) | II (wt. %) |
|---|---|---|
| Blend A (shell) (80% of the candies) | | |
| Sugar (sucrose) | 58.12 | 49.37 |
| Glucose syrup (solids content 80%) | 41.51 | 49.37 |
| Aroma blend from Example of application 5 | 0.17 | 0.25 |
| I-Menthol | 0.10 | — |
| Lemon oil | 0.10 | 0.10 |
| Citric acid | — | 0.91 |
| Total: | 100 | 100 |
| Blend B (core) (20% of the candies) | | |
| High fructose maize syrup (sugar solids | 84.38 | 84.36 |
| content 85%, only 15% water) | | |
| Glycerol | 15.0 | 15.0 |
| Lecithin | 0.02 | 0.02 |
| Cinnamon oil | — | 0.32 |
| Spearmint oil | 0.28 | — |
| Capsaicin | 0.05 | — |
| Vanillyl alcohol n-butyl ether | — | 0.10 |
| Red dye, as 5% aqueous solution | 0.20 | 0.20 |
| Vanillin | 0.07 | — |
| Total | 100 | 100 |

Candies with a liquid/viscous core were produced on the basis of the methods described in U.S. Pat. No. 6,432,441 (Example 1 therein) and those described in U.S. Pat. No. 5,458,894 or U.S. Pat. No. 5,002,791. The two blends A and B were separately processed to form bases for the shell (blend A) or core (blend B). When consumed by affected individuals, the filled throat candies obtained by means of coextrusion were effective against coughing, sore throat and hoarseness.

### Example of Application 10

### Chewing Gum

Chewing gum base K2 consisted of the following ingredients: 28.5% terpene resin, 33.9% polyvinyl acetate (MW=14,000), 16.25% hydrogenated vegetable oil, 5.5% mono- and diglycerides, 0.5% polyisobutene (MW 75,000), 2.0% butyl rubber (isobutene/isoprene copolymer), 4.6% amorphous silicon dioxide (water content approx. 2.5%), 0.05% antioxidant tert.-butylhydroxytoluene (BHT), 0.2% lecithin, and 8.5% calcium carbonate. Chewing gum base K2 and the chewing gum were produced in a similar manner to U.S. Pat. No. 6,986,907.

| | I (wt. %) | II (wt. %) | III (wt. %) |
|---|---|---|---|
| Chewing gum base K2 | 25.30 | 27.30 | 26.30 |
| Sorbitol | 61.48 | 59.48 | 61.80 |
| Glycerol | 2.40 | 2.40 | 2.40 |
| Lecithin | 7.00 | 7.00 | 7.00 |
| Aspartame | 0.14 | 0.14 | 0.14 |
| Encapsulated aspartame | 0.68 | 0.68 | 0.68 |
| Menthol, spray-dried | 0.50 | — | — |
| Cherry aroma, spray-dried | — | 1.20 | — |
| Aroma blend from | 1.50 | 1.80 | — |
| Example of application 4, | | | |
| spray-dried | | | |
| Aroma blend from | 1.00 | — | 1.68 |
| Example of application 3 | | | |

The chewing gums of formulations (I) and (II) were shaped into strips, the chewing gum of formulation (III) was shaped into pellets.

A distinct cooling action could be identified when using the resultant chewing gum.

### Example of Application 11

### Gelatin Capsules for Direct Consumption

| | I (wt. %) | II (wt. %) | III (wt. %) |
|---|---|---|---|
| Gelatin shell: | | | |
| Glycerol | 2.014 | 2.014 | 2.014 |
| Gelatin 240 Bloom | 7.91 | 7.91 | 7.91 |
| Sucralose | 0.065 | 0.065 | 0.065 |
| Allura red | 0.006 | 0.006 | 0.006 |
| Brilliant blue | 0.005 | 0.005 | 0.005 |
| Core composition: | | | |
| Vegetable oil triglyceride | 79.39 | 68.40 | 58.25 |
| (coconut oil fraction) | | | |
| Cinnamon/aniseed aroma | 10.00 | 20.90 | — |
| Eucalyptus aroma | — | — | 29.95 |
| Neotame and aspartame | 0.01 | 0.05 | — |
| Sucralose | 0.22 | 0.30 | 0.70 |
| Aroma blend from Example of | 0.33 | — | — |
| application 5 | | | |
| Aroma blend from Example of | — | 0.20 | 0.60 |
| application 3 | | | |
| L-N^{α}-(Menthanecarboxyl)- | — | 0.05 | — |
| glycine-N-ethylamide from | | | |
| Example 3 | | | |
| (−)-Menthone glycerol acetal | — | 0.10 | 0.40 |
| (Frescolat MGA) | | | |
| Vanillin | 0.05 | — | 0.10 |

Gelatin capsules I, II, III suitable for direct consumption were produced according to WO 2004/050069 and in each case had a diameter of 5 mm, the weight ratio of core material to shell material being 90:10. The capsules in each case opened in the mouth within less than 10 seconds and dissolved completely within less than 50 seconds.

A distinct cooling action could be identified when using the resultant gelatin capsules.

### Example of Application 12

### Chewable Candy

| | | wt. % |
|---|---|---|
| Water | | 7.80% |
| Sugar | Confectioner's sugar C4 | 42.10% |
| Glucose syrup | Dextrose 40 | 37.30% |
| Hardened vegetable fat | Melting point 32-36° C. | 6.60% |
| Lecithin | Emulsifier (soya lecithin) | 0.30% |
| Gelatin | Pig gelatin | 0.80% |
| Fondant | Type - S30 | 4.80% |
| Raspberry aroma | | 0.22% |
| Aroma blend from Example of | | 0.08% |
| application 3 | | |

Manufacturing Instructions:
a) allow gelatin to swell in water (1.8 times the quantity of gelatin) at 70° C. for 2 hours;
b) boil sugar, syrup, water, fat and lecithin at 123° C.;
c) slowly mix gelatin solution with the boiled batch;
d) stir in aroma from Example 2 and optionally color;
e) leave the resultant mass to adjust to approx. 70° C. on a cooling table, then add fondant and aerate for approx. 3 minutes on a pulling machine;
f) then chop and package the chewable candy mass.

When the chewable candy is consumed, a fresh, cooling raspberry flavor is perceived during chewing.

### Example of Application 13

### Extrudate

| | | |
|---|---|---|
| Glucose syrup, spray-dried | Glucidex IT33W (from | 62.0% |
| (DE value: 31-34) | Roquette) | |
| Maltodextrin (DE value: 17-20) | (from Cerestar) | 28.4% |
| Monomuls emulsifier | Emulsifier based on | 1.8% |
| | hardened palm oil; | |
| | melting point: 64° C. | |
| | (from Grünau) | |
| Dextrose monohydrate (DE value: 99.5) | Dextrose, containing | 1.8% |
| | water of crystallization | |
| | (from Cerestar) | |
| Water | | 2.0% |
| Orange/vanilla aroma | | 3.2% |
| Aroma blend from Example of | | 0.8% |
| application 4 | | |

Manufacturing Instructions (See Also WO 03/092412):

All constituents were mixed and conveyed into a twin screw extruder by single point apportionment. Extrusion temperatures were between 100 and 120° C., specific energy input being 0.2 kWh/kg. The strands emerging from the die plate, which is provided with 1 mm holes, were chopped by rotating blades into approx. 1 mm diameter particles immediately on leaving the die.

### Example of Application 14

### Fluidized Bed Granules

A solution consisting of 44 wt. % water, 8 wt. % lemon aroma, 3 wt. % aroma blend from Example of Application 4, 13 wt. % gum arabic and 32 wt. % hydrolyzed starch (Maltodextrin DE 15-19) and a little green dye is granulated in a granulating apparatus of the type presented in EP 163 836 (with the following features: diameter of distributor base plate: 225 mm, spray nozzle: two-fluid nozzle; pneumatic classifying discharge: zig-zag pneumatic classifier; filter: internal bag filter). The solution is sprayed into the fluidized bed granulator at a temperature of 32° C. The bed contents are fluidized by blowing in nitrogen in a quantity of 140 kg/h. The inlet temperature of the fluidizing gas is 140° C. The temperature of the exhaust gas is 76° C. The pneumatic classifying gas used is likewise nitrogen in an amount of 15 kg/h with a temperature of 50° C. The contents of the fluidized bed amounts to approx. 500 g. Granulation output amounts to approx. 2.5 kg per hour. Free-flowing granules are obtained having an average particle diameter of 360 micrometers. The granules are round and exhibit a smooth surface. On the basis of the constant pressure drop of the filter and of the likewise constant bed contents, steady-state conditions may be assumed to prevail with regard to the granulation process.

### Example of Application 15

### Tea Bag with Rooibos or Black Tea and Extrudates from Example of Application 13 or Granules from Example of Application 14

800 g portions of red bush tea (rooibos tea) were mixed in one case with 33 g of the extrudates from Example of application 13 and in one case with 30 g of granules from Example of application 14, portioned and then packaged in tea bags. 800 g portions of black tea (leaf grade: fannings) were mixed in one case with 33 g of the extrudates from Example of application 13 and in one case with 30 g of granules from Example of application 14, portioned and then packaged in tea bags.

The effects found in the Example of application may be transferred, optionally by means of modifications which may straightforwardly be carried out by a person skilled in the art, to any product of the relevant product group, i.e. in particular to toothpastes, chewing gums, mouthwashes, throat candies, gelatin capsules, chewable candies and tea in bags. It will be immediately obvious to a person skilled in the art that the compounds, mixtures and blends according to the invention, optionally with slight modifications, are interchangeable. This means that the compound according to the invention used in the products of the Examples of application must also be considered to represent the other compounds, blends and mixtures according to the invention. The concentration of the compound, blend or mixture according to the invention used may also be varied in a manner obvious to a person skilled in the art. Moreover, the further, product-specific constituents in the particular Example of application may likewise straightforwardly be replaced or supplemented by further typical product constituents by a person skilled in the art. Numerous such product-specific constituents are disclosed in the above-stated description.

What is claimed is:

1. A compound of the formula (I): 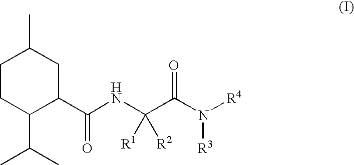
wherein
R¹ and R² in each case mutually independently mean hydrogen or a
(a) linear, branched or cyclic,
(b) saturated or unsaturated,
(c) unsubstituted, mono- or polysubstituted hydrocarbon residue with
(d) 1 to 5 carbon atoms, wherein
(e) the hydrocarbon residues R¹ and R² may also be linked via a single bond or via an —O—, —S— or —NH— group and so preferably form a 3- to 7-membered ring,
and
R³ and R⁴ in each case mutually independently mean hydrogen or a
(a) linear, branched or cyclic,
(b) saturated or unsaturated, hydrocarbon residue with
(c) 1 to 10 carbon atoms, wherein
(d) the hydrocarbon residues R³ and R⁴ may also be linked via a single bond or via an —O—, —S— or —NH— group and so preferably form a 3 to 7-membered heterocyclic ring,
or
R³ and R⁴ mutually independently in each case mean hydrogen or a
(a) linear or branched,
(b) hydrocarbon residue substituted by an R⁵⁻X— group with
(c) 1 to 10 carbon atoms, wherein each X, if present, in each case independently of the other X, if present,
(d) represents oxygen, sulfur or an —NR⁶— group,
and
wherein each R⁵ and each R⁶, if present, in each case mutually independently mean hydrogen or a
(a) linear, branched or cyclic,
(b) saturated or unsaturated, hydrocarbon residue with
(c) 1 to 5 carbon atoms,
and
wherein preferably the particular compound of the formula (I) is present as an individual stereoisomer (enantiomer and diastereomer) or as a specific mixture of different stereoisomers.

2. The compound as claimed in claim 1, wherein in the formula (I)
R¹ is hydrogen
and
R² is hydrogen or a
(a) linear, branched or cyclic,
(b) saturated or unsaturated,
(c) unsubstituted, mono- or polysubstituted hydrocarbon residue with
(d) 1 to 5 carbon atoms
and
R³ and R⁴ in each case mutually independently mean hydrogen or a
(a) linear, branched or cyclic, hydrocarbon residue with
(b) 1 to 10 carbon atoms, wherein
(c) the hydrocarbon residues R³ and R⁴ are preferably linked via a single bond or via an —O—, or —NH— group and so form an azirine, azetidine, pyrrolidine, imidazolidine, piperidine, pyrazolidine, diazane or morpholine ring
or
R³ and R⁴ mutually independently in each case mean hydrogen or a
(a) linear or branched,
(b) hydrocarbon residue substituted by an R⁵⁻X— group with
(c) 1 to 10 carbon atoms, wherein preferably each X, if present, in each case independently of the other X, if present,
(d) represents oxygen, sulfur or an —NR⁶— group and wherein preferably each R⁶, if present, means hydrogen.

3. The compound as claimed in claim 1, wherein the compound of the formula (I) is 90-100 mol % defined, with regard to the stereochemistry thereof, in accordance with the formula (Ia) 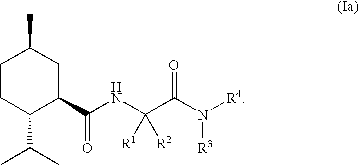
wherein
in the formula (Ia) residues R¹, R², R³ and R⁴ in each case have the meaning of the corresponding residues of the formula (I).

4. The compound as claimed in claim 3, wherein in the formulae (I) and (Ia)
R¹ is hydrogen
and
R² is hydrogen, methyl, ethyl, propyl, 2-propyl, 2-methylpropyl, 2-butyl, 2-methylbutyl, 4-aminobutyl, 3-guanidinopropyl, 3-aminopropyl, 3-ureidopropyl, indol-3-ylmethyl, 2-carboxyethyl, carboxymethyl, 2-(aminocarbonyl)ethyl, (aminocarbonyl)methyl, thiomethyl, 2-thioethyl, 2-methylthioethyl, hydroxymethyl, 1-hydroxyethyl, phenylmethyl or 4-hydroxyphenylmethyl residue
and
R³ is hydrogen, methyl or ethyl residue,
and
R⁴ is hydrogen, methyl, ethyl, 2-propyl, cyclopropyl, propyl, butyl, cyclobutyl, 2-butyl, 2-methylprop-1-yl, 2-methylprop-2-yl, pentyl, cyclopentyl, 2-pentyl, 3-pentyl, hexyl, cyclohexyl, 2-hexyl and 3-hexyl, heptyl, octyl, isooctyl, nonyl or decyl residue,
or
R⁴ is methylene, ethylene, 1,2-propylene, 1,3-propylene, 1,2-butylene, 1,3-butylene, 1,4-butylene, 1,2-pentylene, 1,3-pentylene, 1,4-pentylene or 1,5-pentylene residue substituted by an R⁵⁻X— group, wherein X represents oxygen and R⁵ is a hydrogen, methyl or ethyl residue.

5. The compound as claimed in claim 3, wherein in the formulae (I) and (Ia)
R² is hydrogen, methyl, ethyl, propyl or 2-propyl residue
and
R⁴ is hydrogen, methyl, ethyl, 2-propyl, cyclopropyl, propyl, butyl, cyclobutyl, 2-butyl, 2-methylprop-1-yl, 2-methylprop-2-yl, pentyl, cyclopentyl, 2-pentyl, 3-pentyl, hexyl, cyclohexyl, 2-hexyl and 3-hexyl, heptyl, octyl, isooctyl, nonyl or decyl residue
or
R⁴ is a methylene, ethylene, 1,2-propylene or 1,3-propylene residue substituted by an R⁵⁻X— group, wherein X represents oxygen, and
R⁵ is hydrogen or a methyl residue.

6. The compound as claimed in claim 1, wherein said compound of the formula (I)
is L-N^{α}-(menthanecarboxyl)glycine-N-isobutylamide,
L-N^{α}-(menthanecarboxyl)glycine-N,N-dimethylamide,
L-N^{α}-(menthanecarboxyl)glycine-N-ethylamide,
L-N^{α}-(menthanecarboxyl)glycine-N-ethanolamide or
L-N^{α}-(menthanecarboxyl)-L-alanine-N-ethylamide.

7. A mixture comprising:
(a) a compound of the formula (I): 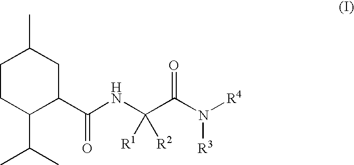
wherein
R¹ and R² in each case mutually independently mean hydrogen or a
(a) linear, branched or cyclic,
(b) saturated or unsaturated,
(c) unsubstituted, mono- or polysubstituted hydrocarbon residue with
(d) 1 to 5 carbon atoms, wherein
(e) the hydrocarbon residues R¹ and R² may also be linked via a single bond or via an —O—, —S— or —NH— group and so preferably form a 3- to 7-membered ring,
and
R³ and R⁴ in each case mutually independently mean hydrogen or a
(a) linear, branched or cyclic,
(b) saturated or unsaturated, hydrocarbon residue with
(c) 1 to 10 carbon atoms, wherein
(d) the hydrocarbon residues R³ and R⁴ may also be linked via a single bond or via an —O—, —S— or —NH— group and so preferably form a 3 to 7-membered heterocyclic ring,
or
R³ and R⁴ mutually independently in each case mean hydrogen or a
(a) linear or branched,
(b) hydrocarbon residue substituted by an R⁵⁻X— group with
(c) 1 to 10 carbon atoms, wherein each X, if present, in each case independently of the other X, if present,
(d) represents oxygen, sulfur or an —NR— group,
and
wherein each R⁵ and each R⁶, if present, in each case mutually independently mean hydrogen or a
(a) linear, branched or cyclic,
(b) saturated or unsaturated, hydrocarbon residue with
(c) 1 to 5 carbon atoms,
and
wherein preferably the particular compound of the formula (I) is present as an individual stereoisomer (enantiomer and diastereomer) or as a specific mixture of different stereoisomers;
(b) one or more further substances having a physiological cooling action;
(c) one or more aroma substances not having a physiological cooling action;
and
(d) one or more substances without a physiological cooling action having a trigeminal or salivatory action.

8. The mixture as claimed in claim 7, further comprising:
one or more compounds which mutually independently or jointly additionally cause a flavor-modulating effect and/or a trigeminal and/or a salivatory stimulus.

9. A preparation which is consumed for nutrition, pleasure, oral hygiene, a pharmaceutical, or a cosmetic comprising a sufficient quantity for achieving a physiological cooling action on the skin and mucous membrane of:
(a) a compound of the formula (I): 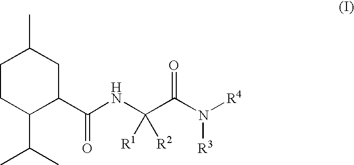
wherein
R¹ and R² in each case mutually independently mean hydrogen or a
(a) linear, branched or cyclic,
(b) saturated or unsaturated,
(c) unsubstituted, mono- or polysubstituted hydrocarbon residue with
(d) 1 to 5 carbon atoms, wherein
(e) the hydrocarbon residues R¹ and R² may also be linked via a single bond or via an —O—, —S— or —NH— group and so preferably form a 3- to 7-membered ring,
and
R³ and R⁴ in each case mutually independently mean hydrogen or a
(a) linear, branched or cyclic,
(b) saturated or unsaturated, hydrocarbon residue with
(c) 1 to 10 carbon atoms, wherein
(d) the hydrocarbon residues R³ and R⁴ may also be linked via a single bond or via an —O—, —S— or —NH— group and so preferably form a 3 to 7-membered heterocyclic ring,
or
R³ and R⁴ mutually independently in each case mean hydrogen or a
(a) linear or branched,
(b) hydrocarbon residue substituted by an R⁵⁻X— group with
(c) 1 to 10 carbon atoms, wherein each X, if present, in each case independently of the other X, if present,
(d) represents oxygen, sulfur or an —NR⁶— group,
and
wherein each R⁵ and each R⁶, if present, in each case mutually independently mean hydrogen or a
(a) linear, branched or cyclic,
(b) saturated or unsaturated, hydrocarbon residue with
(c) 1 to 5 carbon atoms,
and
wherein preferably the particular compound of the formula (I) is present as an individual stereoisomer (enantiomer and diastereomer) or as a specific mixture of different stereoisomers.

10. A method of producing a cooling action on the skin or mucous membrane for purposes other than therapeutic or for producing a treatment with a mixture comprising:
(a) a compound of the formula (I): 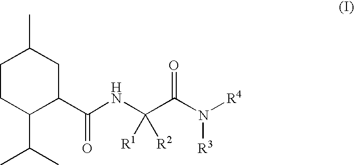
wherein
R¹ and R² in each case mutually independently mean hydrogen or a
(a) linear, branched or cyclic,
(b) saturated or unsaturated,
(c) unsubstituted, mono- or polysubstituted hydrocarbon residue with
(d) 1 to 5 carbon atoms, wherein
(e) the hydrocarbon residues R¹ and R² may also be linked via a single bond or via an —O—, —S— or —NH— group and so preferably form a 3- to 7-membered ring,
and
R³ and R⁴ in each case mutually independently mean hydrogen or a
(a) linear, branched or cyclic,
(b) saturated or unsaturated, hydrocarbon residue with
(c) 1 to 10 carbon atoms, wherein
(d) the hydrocarbon residues R³ and R⁴ may also be linked via a single bond or via an —O—, —S— or —NH— group and so preferably form a 3 to 7-membered heterocyclic ring,
or
R³ and R⁴ mutually independently in each case mean hydrogen or a
(a) linear or branched,
(b) hydrocarbon residue substituted by an R⁵⁻X— group with
(c) 1 to 10 carbon atoms, wherein each X, if present, in each case independently of the other X, if present,
(d) represents oxygen, sulfur or an —NR— group,
and
wherein each R⁵ and each R⁶, if present, in each case mutually independently mean hydrogen or a
(a) linear, branched or cyclic,
(b) saturated or unsaturated, hydrocarbon residue with
(c) 1 to 5 carbon atoms,
and
wherein preferably the particular compound of the formula (I) is present as an individual stereoisomer (enantiomer and diastereomer) or as a specific mixture of different stereoisomers.

11. The method as claimed in claim 10, wherein the medicament combats or alleviates coughs, colds, symptoms of oral, nasal, throat or pharyngeal inflammation, sore throat or hoarseness.

12. A method for achieving a physiological cooling action on the skin and/or a mucous membrane comprising the step of:
applying onto the skin or mucous membrane a quantity sufficient for achieving said physiological cooling action, a mixture comprising:
(a) a compound of the formula (I): 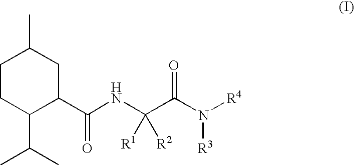
wherein
R¹ and R² in each case mutually independently mean hydrogen or a
(a) linear, branched or cyclic,
(b) saturated or unsaturated,
(c) unsubstituted, mono- or polysubstituted hydrocarbon residue with
(d) 1 to 5 carbon atoms, wherein
(e) the hydrocarbon residues R¹ and R² may also be linked via a single bond or via an —O—, —S— or —NH— group and so preferably form a 3- to 7-membered ring,
and
R³ and R⁴ in each case mutually independently mean hydrogen or a
(a) linear, branched or cyclic,
(b) saturated or unsaturated, hydrocarbon residue with
(c) 1 to 10 carbon atoms, wherein
(d) the hydrocarbon residues R³ and R⁴ may also be linked via a single bond or via an —O—, —S— or —NH— group and so preferably form a 3 to 7-membered heterocyclic ring,
or
R³ and R⁴ mutually independently in each case mean hydrogen or a
(a) linear or branched,
(b) hydrocarbon residue substituted by an R⁵⁻X— group with
(c) 1 to 10 carbon atoms, wherein each X, if present, in each case independently of the other X, if present,
(d) represents oxygen, sulfur or an —NR⁶— group,
and
wherein each R⁵ and each R⁶, if present, in each case mutually independently mean hydrogen or a
(a) linear, branched or cyclic,
(b) saturated or unsaturated, hydrocarbon residue with
(c) 1 to 5 carbon atoms,
and
wherein preferably the particular compound of the formula (I) is present as an individual stereoisomer (enantiomer and diastereomer) or as a specific mixture of different stereoisomers.

13. A method for the producing a compound of the formula (I): 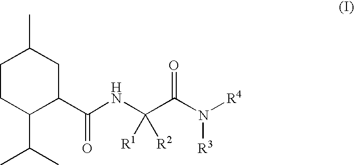
wherein
R¹ and R² in each case mutually independently mean hydrogen or a
(a) linear, branched or cyclic,
(b) saturated or unsaturated,
(c) unsubstituted, mono- or polysubstituted hydrocarbon residue with
(d) 1 to 5 carbon atoms, wherein
(e) the hydrocarbon residues R¹ and R² may also be linked via a single bond or via an —O—, —S— or —NH— group and so preferably form a 3- to 7-membered ring,
and
R³ and R⁴ in each case mutually independently mean hydrogen or a
(a) linear, branched or cyclic,
(b) saturated or unsaturated, hydrocarbon residue with
(c) 1 to 10 carbon atoms, wherein
(d) the hydrocarbon residues R³ and R⁴ may also be linked via a single bond or via an —O—, —S— or —NH— group and so preferably form a 3 to 7-membered heterocyclic ring,
or
R³ and R⁴ mutually independently in each case mean hydrogen or a
(a) linear or branched,
(b) hydrocarbon residue substituted by an R⁵⁻X— group with
(c) 1 to 10 carbon atoms, wherein each X, if present, in each case independently of the other X, if present,
(d) represents oxygen, sulfur or an —NR— group,
and
wherein each R⁵ and each R⁶, if present, in each case mutually independently mean hydrogen or a
(a) linear, branched or cyclic,
(b) saturated or unsaturated, hydrocarbon residue with
(c) 1 to 5 carbon atoms,
and
wherein preferably the particular compound of the formula (I) is present as an individual stereoisomer (enantiomer and diastereomer) or as a specific mixture of different stereoisomers
comprising the steps of:
(a) providing a corresponding N^{α}-(menthanecarbonyl)amino acid, a corresponding N^{α}-(menthanecarbonyl)amino acid chloride or another corresponding activated N^{α}-(menthanecarbonyl)amino acid derivative,
(b) providing a corresponding amine or a corresponding salt and
(c) reacting the provided compounds with one another.

14. The preparation as claimed in claim 9, further comprising:
(a) one or more further substances having a physiological cooling action;
(b) one or more aroma substances not having a physiological cooling action;
and
(c) one or more substances without a physiological cooling action having a trigeminal or salivatory action.

15. The method of claim 10, wherein said mixture further comprises:
(a) one or more further substances having a physiological cooling action;
(b) one or more aroma substances not having a physiological cooling action;
and
(c) one or more substances without a physiological cooling action having a trigeminal or salivatory action.

16. The method of claim 12, wherein said mixture further comprises:
(a) one or more further substances having a physiological cooling action;
(b) one or more aroma substances not having a physiological cooling action;
and
(c) one or more substances without a physiological cooling action having a trigeminal or salivatory action.
